(19) United States
(12) Patent Application Publication
(10) Pub. No.: US 2019/0365559 A1
(43) Pub. Date: Dec. 5, 2019

(54) VALVE ASSEMBLY AND PENIS PUMP

(71) Applicant: ULTRAMAX PRODUCTS LTD, Wakefield West Yorkshire (GB)

(72) Inventor: John Oakes, Wakefield, West Yorkshire (GB)

(21) Appl. No.: 16/477,835

(22) PCT Filed: Jan. 12, 2018
(86) PCT No.: PCT/GB2018/050089

§ 371 Date: Jul. 12, 2019

(30) Foreign Application Priority Data
Jan. 13, 2017 (GB) 1700647.9

(51) Int. Cl. A61F 5/41

(52) U.S. Cl. CPC A61F 5/41; A61F 2005/412

(57) ABSTRACT

A valve assembly (100) for a penis pump (1102), comprises a valve cap (110) comprising a hole (115); and a valve stem (120) extending through the hole (115) in the valve cap (110). The valve assembly (100) is adapted such that axial motion of the valve stem (120) causes the valve stem (120) to mechanically interact with the valve cap (110), so that the valve stem (120) moves between a first position, in which the valve stem (120) seals a pump chamber (1108) of the penis pump (1102), and a second position, in which the valve stem (120) operates as a one way valve. Accordingly, axial motion of the valve stem (120) is used to select the function of the valve assembly (100).

### FIELD

This invention relates to a valve assembly for a penis pump, and a penis pump comprising the valve.

### BACKGROUND

Penis pumps, which cause or enhance an erection of a penis, particularly a human penis, have been known in the art for some years. The manner in which such pumps work is by placing a chamber over a flaccid penis and evacuating the chamber. The evacuation causes a pressure differential between the inside and outside of the chamber. The lower pressure within the chamber causes blood to flow into the penis and thus make the penis erect.

WO 2006/024874 and WO 2011/135374 disclose pumps that address many of the problems of the prior art and provide devices that induce a strong and enduring erection. Such devices, in which the evacuated medium envisaged is liquid, not gas, are generally referred to as hydropump penis pumps. Conventional penis pumps in which the evacuated medium is gas are generally referred to as vacuum penis pumps.

Typically, both hydropump and vacuum penis pumps comprise a one way valve (also known as a non-return valve), which is used in evacuating the chamber to reduce the pressure therein.

The invention relates to a new valve design, particularly but not exclusively to overcome several manufacturing and practical issues currently found in the manufacture of the valve systems of hydropump/vacuum pumps currently found on the market today.

In principle the invention supersedes the previous one way valve designs which would also incorporate a closed latch feature that would allow the valve to be locked closed thus stopping the flow of the air or fluid such as water from passing the valve seal. An example of a previous one way valve design 10 is shown in FIGS. 1a and 1b. The previous valves 10 would have to be manufactured in a 2 part over mould material process whereupon the harder base material (which forms the structure of the valve base and the latch mechanism, and is generally indicated by reference numeral 11) would first be moulded by normally an injection moulding process. Then a far softer material (which would form the seal of the sealing mechanism, and is generally indicated by reference numeral 12) would be secondary over moulded to the first hard part material.

The part 10 is the stem of the valve assembly, which acts as the valve and which would be disposed in a housing (not shown) in use. The above-referenced closed latch feature involves pressing the stem laterally with a force A, as shown in FIG. 1a.

The process for producing the valve 10 would involve using two injection moulding tools at great expense. Due to the high melt temperatures that are need for the over moulding material to process and the need for mechanical adhesion of the two material parts. Through intense trials it was found that the injection moulding processing parameters were indeed very small and needed to be consistently monitored and regular inspection necessary to ensure that the part would be correctly formed without heat distortion or deformation due to the high melt temperatures and high compression forces normally associated with the injection moulding process. Inspection would normally be done by a trained inspector looking at the formation of the soft seal 12 though the magnifying glass or microscope and placing the valve part in a pressure test machine whereupon the valve would be tested for low pressure leaks. All of the above would involve expensive machinery and man hours to consistently produce workable valves. The valve 10 would act as a one way valve and a push over latch locking valve but had no ability to remain open thus allowing the air or fluid such as water to pass in both directions in and out of the vacuum chamber.

It is an aim of the invention to overcome the above-mentioned difficulties, and any other difficulties that would be apparent to the skilled reader from the description herein. It is a further aim of the invention to provide a valve for a penis pump that is cost effective to manufacture and which enables easy control of the pressure in the pump chamber.

### SUMMARY

According to the present invention there is provided an apparatus and method as set forth in the appended claims. Other features of the invention will be apparent from the dependent claims, and the description which follows.

According to a first aspect of the invention there is provided a valve assembly for a penis pump, comprising:
a valve cap attachable to the penis pump and comprising a hole; and
a valve stem extending through the hole in the valve cap, the valve assembly being configured such that axial motion applied to the valve stem causes the valve stem to mechanically interact with the valve cap, so that the valve stem moves between:
   a first position, in which the valve stem is retained so as to seal a pump chamber of the penis pump, and
   a second position, in which the valve stem operates as a one way valve.

The valve assembly may permit egress of fluid from the pump chamber in the second position. The valve assembly may permit egress of fluid when pressure inside the pump chamber exceeds the pressure outside the pump chamber. The valve assembly may prevent ingress of fluid to the pump chamber in the second position. References to fluids herein encompass both liquids (including water) and gases (including air).

The valve stem may comprise at least one stem projection formed thereon. The at least one stem projection may be a ridge. The ridge may be annular. The at least one stem projection may interfere with the valve cap. The at least one stem projection may deform to allow the valve stem to move between the positions.

The hole may comprise a hole projection to mechanically interact with, preferably interfere with, the valve stem. The hole projection may mechanically interact with the at least one stem projection of the valve stem. The hole projection may be annular. The hole projection may be formed at or toward one end of the hole, preferably the end proximate to the pump chamber in use. The hole projection may be a lip formed at one end of the hole.

The at least one stem projection may include a first projection. In the first position, the first projection may be disposed at a side of the hole projection that is proximal to the pump chamber. In the second position, the first projection may be disposed at side of the hole projection that is distal from the pump chamber.

The valve assembly may be adapted such that axial motion of the valve stem causes the valve stem to move to a third position, in which the valve assembly is locked open. The at least one stem projection may include a second projection. The second projection may be disposed closer to the pump chamber in use than the first projection. In the third position, the second projection may be disposed at the side of the hole projection that is distal from the pump chamber.

The valve stem may comprise a flange for sealing the pump chamber. The flange may extend radially from the valve stem. The flange may be resiliently deformable. The flange may be configured to deform in the first position.

The valve stem may be flexible. The valve stem may comprise or be formed of a thermoplastic elastomeror silicone material.

The valve cap may be rigid. The valve cap may comprise or be formed of acetal, also known as Polyoxymethylene (POM). The valve cap may comprise or be formed of metal or alloy.

The valve assembly may be configured so that, in the second position, receipt of a radial force applied to the stem causes the seal of pump chamber to be partially opened. Accordingly, the partial opening of the seal allows a slow release of low pressure from inside the pump chamber.

The valve assembly may comprise a valve body. The valve body may have a first portion arranged to receive the valve cap, and a second portion sealable by the valve stem, preferably the flange of the valve stem. The second portion may have a smaller diameter than the first portion.

Solely axial motion may be sufficient to move the valve stem between the first and second positions, and/or between the second position and third position.

The valve assembly may be suitable for a hydropump penis pump.

According to a second aspect of the invention there is provided a penis pump comprising the valve assembly of the first aspect.

The penis pump may comprise a pump chamber adapted to receive a human penis, a base section arranged at a first end of the chamber for sealing around the human penis, and pumping means operable to pump fluid from the pump chamber.

The penis pump may comprise a head section at a second end of the chamber, which may comprise the valve assembly.

The base section may comprise the pumping means. The pumping means may comprise a compressible gaiter, biased to return to an uncompressed configuration.

The pump may be a hydropump, adapted to pump liquid from the chamber.

According to a third aspect of the invention there is provided there is provided a method of causing or enhancing an erection of a human penis comprising:

inserting a generally flaccid penis into a chamber of a penis pump and using pumping means to pump water from the chamber through a valve assembly as defined in the first aspect.

The method may comprise moving the valve stem of the valve assembly to the second position.

The penis pump may be the penis pump of the second aspect.

The method of the third aspect may be combined with any features of the first or second aspects, or any other feature described herein, in any combination.

Further aspects are detailed below.

In principle the invention is a one way valve, designed to open a limited amount to allow air or fluid such as water to pass in the outwards direction forced by the compression of the bellows pump system below the pump chamber. Alternatively, by vacuum generated by the various types of hand held vacuum pump systems which would attach to the end of the hydropump/vacuum chamber, thus removing the air or fluid from inside the chamber.

In principle the invention is a new type push/pull one way valve which overcomes the need for the two part hard and soft material over moulding and the need for individual tools for the hard base material valve part body or the secondary over mould part. It removes the manufacture of the over mould injection tool as it uses a different moulding process that has one tool and one reasonably soft material and in its simplistic design and provides the ability to work in three essential modes: as a one way valve, as a push down lock valve and also as a pull open stay open valve. The simple design uses several positioned ridges along the valve upper stem to mechanically interact against the valve cap in three positions.

The Push Down Lock Position

In principle the invention push down lock position is where the valve is held down by the stem upper ring profile and mechanically held by the valve cap underside formed profile. The tension in the valve material counteracts the vacuum pressure inside the vacuum chamber and creates a well formed positive seal against the valve body inner hole to seal the flow of air or fluid such as water from passing in both directions in the valve body hole. The mechanical spring properties of the valve material act as a sprung tensioner wanting to counteract the down wards force applied by the user, thus giving the valve the positive locking feel and action.

The One Way Valve Position

In principle the invention one way valve position operates when the valve stem is positioned in the midpoint thus allowing the valve to move between the two ring profiles and seal on to the valve body hole, thus stopping the flow of air or fluid such as water or opening and letting pass the above air or fluids when positive low pressure is forcing the valve open. But when the pressure is reduced to a point or reversed the valve would seal as a gravity valve and stop the flow of air or fluids such as water from passing.

The Pull Open Lock Position

In principle the invention pull open lock valve position is when the user pinches the stem between the thumb and first finger and applies sufficient pressure to pull the stem lower ring profile past the valve cap underside and upper profile thus locking the valve open. This action allows the passing of air or fluid such as water to pass in both the inward and outwards direction of the vacuum chamber. The user can then place the thumb over the stem of the valve and apply a slight down wards force to push the valve in to the one way valve position or further downwards pressure to the valve locking it in the lock down.

### BRIEF DESCRIPTION OF DRAWINGS

For a better understanding of the invention, and to show how embodiments of the same may be carried into effect, reference will now be made, by way of example only, to the accompanying diagrammatic drawings in which:

FIGS. 1a and 1 b are respectively side and perspective views of a prior art valve;

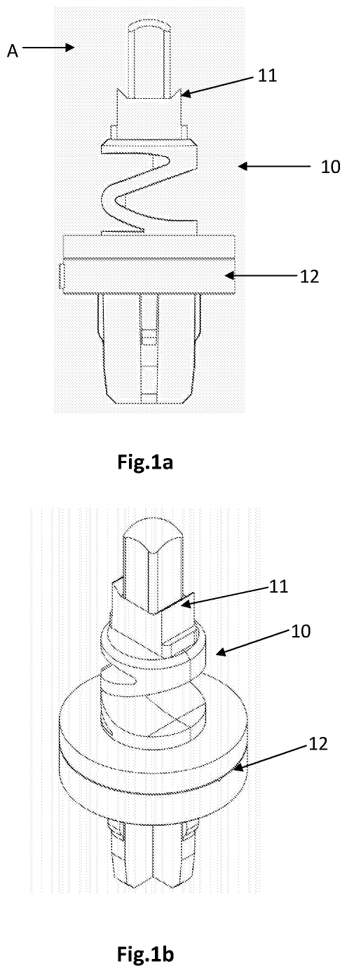

FIG. 2 is a side view of an exemplary penis pump;

FIGS. 3a and 3b are respectively perspective and side views of an exemplary valve assembly;

FIGS. 4a and 4b are respectively perspective and side views of a valve stem of the valve assembly of FIGS. 3a and 3b;

FIG. 5 is a cross-sectional view showing the valve assembly of FIGS. 3 and 4 in a locked open position;

FIG. 6 is a cross-sectional view showing the valve assembly of FIGS. 3 to 5 in a one way valve position;

FIG. 7 is a cross-sectional view showing the valve assembly of FIGS. 3 to 6 in a locked closed position; and

FIG. 8 a cross-sectional view showing the valve assembly of FIGS. 3 to 7 in a slow release position.

In the drawings, corresponding reference characters indicate corresponding components. The skilled person will appreciate that elements in the figures are illustrated for simplicity and clarity and have not necessarily been drawn to scale. For example, the dimensions of some of the elements in the figures may be exaggerated relative to other elements to help to improve understanding of various example embodiments. Also, common but well-understood elements that are useful or necessary in a commercially feasible embodiment are often not depicted in order to facilitate a less obstructed view of these various example embodiments.

### DESCRIPTION OF EMBODIMENTS

In overview, examples of the invention provide a valve assembly for a penis pump, which comprises a stem and a valve cap. The stem and valve cap interact when the stem is moved axially, such that the valve assembly can be retained in a first position, in which the pump chamber is locked closed and a second position, in which the valve acts as a one way valve. In some examples, the valve assembly can also be retained in a third position, in which the valve is locked open, and/or the valve stem may be tilted to effect a slow release of pressure.

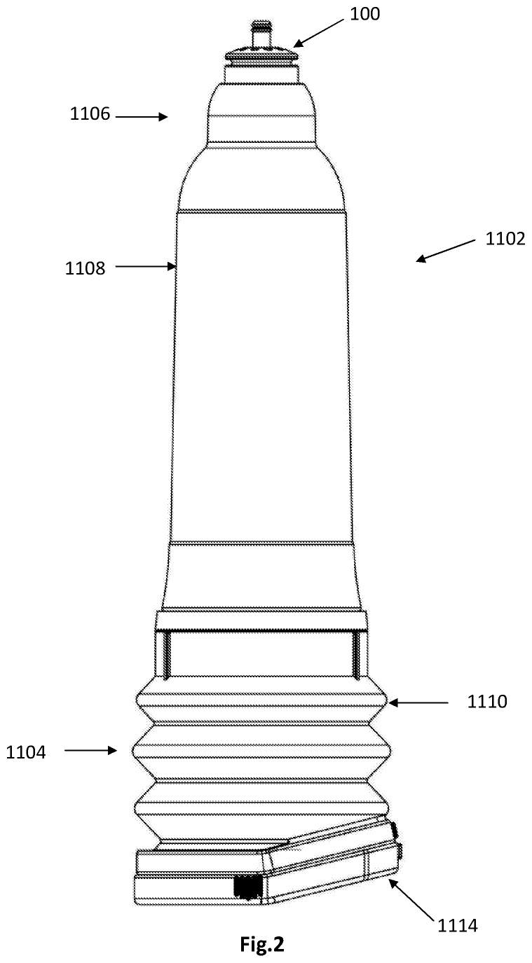

FIG. 2 shows an exemplary penis pump 1102. The pump 1102 has a cylindrical chamber 1108 which is generally hollow, a base section 1104 at a first end thereof and a head section 1106 at a second end thereof.

The base section 1104 comprises a rubber gaiter 1110 which connects at an upper end thereof to the cylindrical chamber 1108. It will be appreciated that the chamber 1108 need not be strictly cylindrical, and may instead taper slightly from the base section 1104 to the head section 1106. The connection between the gaiter 1110 and the chamber 1108 allows relative rotation there between, thus enabling the lower portion of the pump to be rotated while the chamber is static. This allows a user to rotate the gaiter 1110 in use, thus allowing easy movement between different pump orientations. This is particularly advantageous when the pump is being used as an aid to erectile dysfunction, especially in conjunction with a constriction ring (not shown).

The base section 1104 has a chamfer 1114 at one side thereof thus allowing the gaiter 1100 to accommodate the testicles of a user in one arrangement, or to be arranged in an acute angle to the body of a user in an alternative arrangement.

The head section 1106 comprises a valve assembly 100, which will be discussed in detail below. The valve assembly 100 is accommodated in a recess (not shown) formed at the tip of the head section 1106.

Use of the penis pump 1102 will now be briefly described.

In use, the gaiter 1110 may be compressed in a concertina type action, thus decreasing the volume inside the pump. The gaiter 1110 has spring properties which restore its compressed state back to its uncompressed state. Accordingly, compression of the gaiter 1110 causes fluid to be expelled from the chamber via the valve assembly 100.

In order to use the pump to produce an erection to a human penis, the pump 1102 and a user may be immersed in liquid, such as in a bath, hot tub or jacuzzi. Alternatively, the device may be used in air. In one arrangement, the device may be filled with liquid, such as in a shower scenario, but the user not necessarily immersed in liquid. The flaccid or semi erect penis is then placed into the chamber 1108 via the base section 1104. The base section 1104 is pulled down so that it abuts the user's pubic area and forms a seal. The chamfer 1114 is arranged either against the testicular region to provide safety and comfort, or the chamfer 1114 may be arranged at the pubic region of the user and the device arranged at an acute angle to the body of the user. The user then pulls the pump toward the body thus causing the gaiter 1110 to compress. Fluid within the chamber is thereby expelled through the valve assembly 100 (depending on the position of components of the valve assembly 100 as discussed below) of the head section 1106, because the volume of the chamber 1108 is decreased. The spring return force of the gaiter 1110 attempts to restore the pump to its original internal volume and thereby reduces the pressure inside the chamber 1108. Continuing the use, the gaiter 1110 may be compressed again to expel more fluid through the valve assembly 100. The penis is thereby encouraged to expand (by the ingress of blood) in order to return the gaiter 1110 to its uncompressed state.

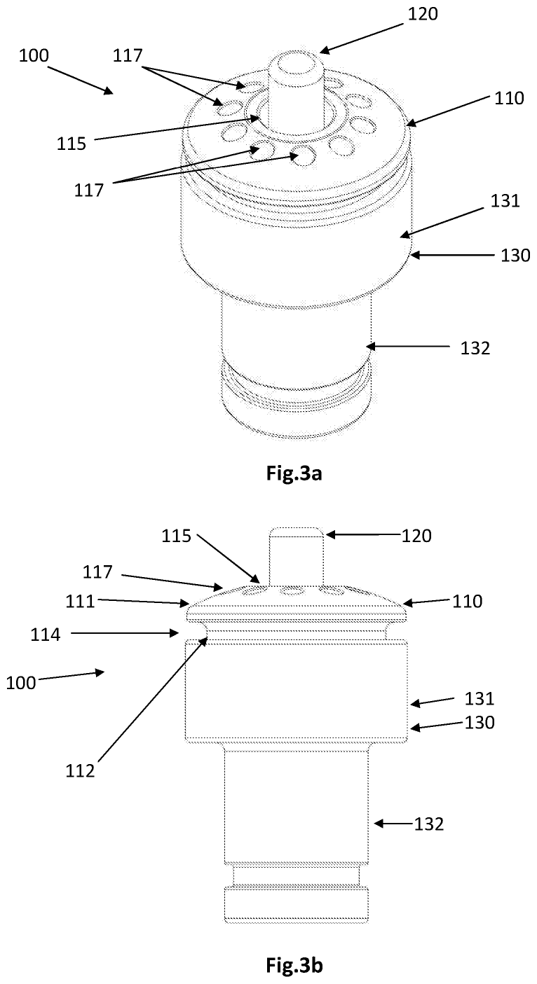

Turning now to FIGS. 3 to 9, the valve assembly 100 will be discussed in more detail. The valve assembly 100 comprises a valve cap 110, a valve stem 120 and a valve body 130.

The valve body 130 takes the form of a stepped cylindrical tube, with wider portion 131 disposed distally from the pump chamber 1108 once installed in the head section 1106, and narrower portion 132 disposed proximate the pump chamber 1108. The valve body 130 retains the valve cap 110 and valve stem 120.

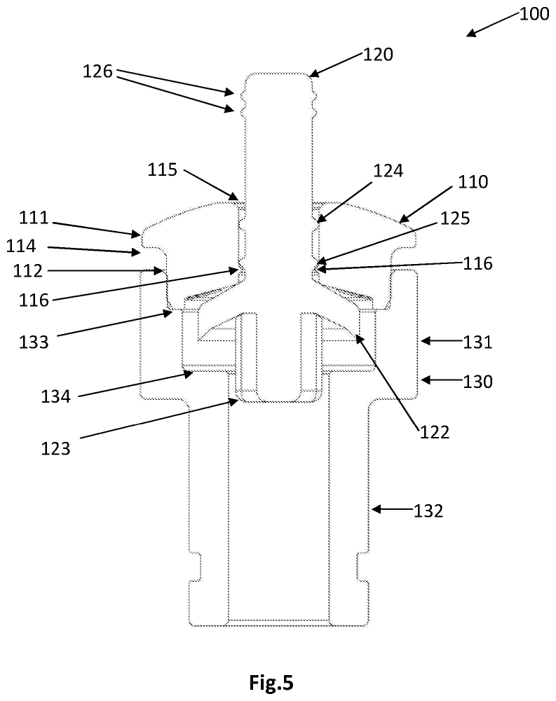

The valve cap 110 takes the form of a stepped cylinder, with its wider portion 111 disposed distally from the pump chamber 1108 once installed in the head section 1106, and narrower portion 112 disposed proximate the pump chamber 1108. The narrower portion 112 engages a lip 133 formed in the interior of the valve body, which securely retains it in place, for example with a snap-fit connection (see FIGS. 5 to 9). A gap 114 is formed between the upper surface of the valve body 130 and the wider portion 111 of the cap 110, which facilitates the removal of the cap 110, for example for cleaning purposes.

The valve cap 110 comprises a hole 115 extending through the valve cap 110 from its upper surface to its lower surface. The hole 115 is disposed centrally within the valve cap 110. In other words, the hole 115 coincides with a central, longitudinal axis of the cylinder of the cap 110. The hole 115 is arranged to receive the valve stem 120. An annular projection 116 (see FIGS. 5 to 9) is formed in the interior of the hole 115, for engagement with the stem 120.

The valve cap 110 also comprises a plurality of holes 117 disposed radially around the central hole 115, through which fluid (e.g. air or water) may pass to exit/enter the cap 110.

The valve cap 110 is formed from a relatively rigid, inflexible plastic. In one example, the valve cap 110 is formed of acetal, also known as Polyoxymethylene (POM). In an alternative example, the valve cap 110 may be formed of a metal or alloy, for example titanium. The valve body 130 is also formed from a relatively rigid, inflexible plastic, which may be the same or a similar material as the valve cap 110.

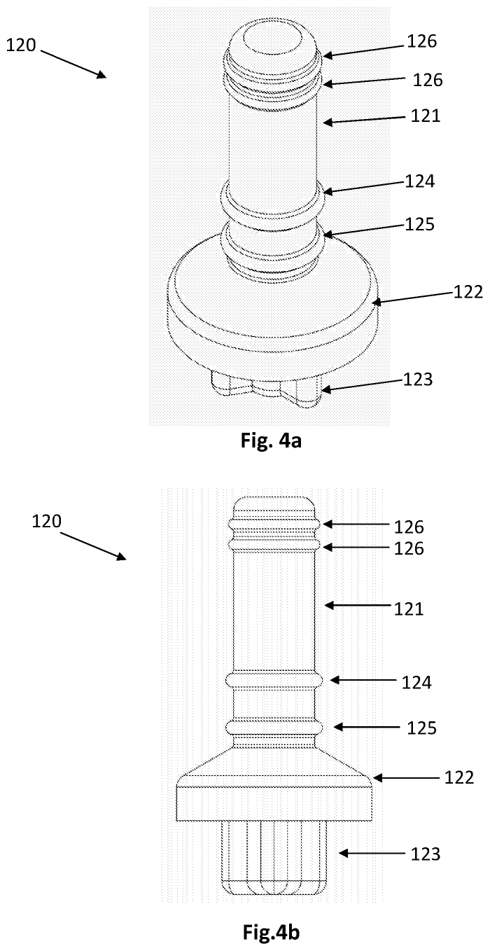

The valve stem 120, which is best seen in FIGS. 4a and 4b, comprises a substantially cylindrical body 121. A flange 122 is arranged around a lower part of the stem 120, with an engagement portion 123 disposed below the flange 122. The flange 122 and the engagement portion 123 are adapted to seal over the entrance to the narrower portion 132 of valve body 130, and thus provides the valve functionality of the valve assembly 100. In particular, peripheral portions of the flange 122 contact a lip 134 formed by the step between the wider portion 131 and narrower portion 132 to form the seal. In addition, the flange 122 retains the valve stem 120 within the valve assembly 100, because its radial extent is greater than the width of hole 115.

The valve stem 120 comprises a first ridge 124 and a second ridge 125, which are disposed annularly about the cylindrical body 121. First ridge 124 is disposed above (i.e. further from the flange 112 than) second ridge 125. In other words, the ridges 124/125 are positioned ridges along the valve upper stem 120. As discussed below, the ridges 124/125 mechanically interact against the valve cap in three positions

Further ridges 126 are disposed around the top end of the stem 120, to facilitate gripping by the user.

The stem 120 is formed from a relatively flexible material, such as a thermoplastic elastomer or a silicone material. Accordingly, whilst the projection 116 normally interferes with ridges 124, 125 to restrict movement of the stem 120, if sufficient force is applied by a user the ridges 124, 125 will deform so as to pass over the projection 116. Accordingly an axial force along a longitudinal axis of the stem 120 by a user (i.e. a push or a pull) changes the position at which the stem 120 is retained. This allows the stem 120 to be set in several positions, which are outlined below.

FIG. 5 shows the valve assembly 100 in a locked open position. The valve stem 120 is positioned such that the second ridge 125 is above the projection 116. It will be appreciated that “above” in this context refers to the ridge 125 being further from the pump chamber 1108 and narrower portion 132 than the projection 116. Accordingly, the flange 122 and engaging portion 123 are spaced apart from the entrance to the narrower portion 132 of the body 130. This therefore permits both egress and ingress of liquid and gas from/to the pump chamber 1108.

The locked open position is of particular benefit if, in use, a layer of air is trapped between layers of water. Such trapped air may occur if a stimulating or training insert (not shown) is provided in the penis pump 1102. The insert is generally cylindrical and comprises an internal cavity and operable to fit within a circumferential ring of the gaiter 1110. The pump chamber 1108 may be filled with warm water with the valve assembly 100 in the closed position before the insert is attached, which allows the insert to be heated to give true sexual feeling for the user. However, if the chamber 1108 is not accurately filled, air may become trapped during the insertion of the cylindrical insert, which causes a piston effect during use. The piston effect can be resolved by selecting the locked open position, allowing the water to exit the chamber 1108. In addition, the locked open position may be useful in cleaning the pump 1102.

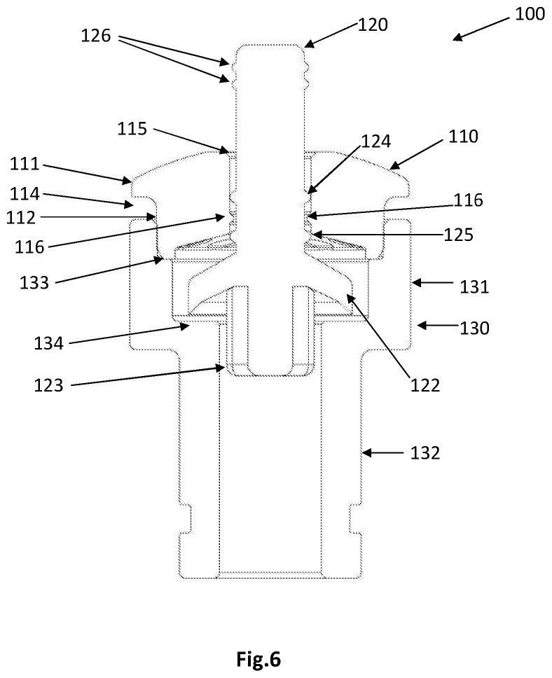

FIG. 6 shows the valve assembly 100 in the one way valve position. In this position, the projection 116 is disposed between the two ridges 124, 125. Accordingly, the valve stem 120 is permitted to move axially between positions in which one ridge 124 and the other ridge 125 contact the projection 116. Accordingly, the flange 122 may seal over the entrance to the narrower portion 132, thus stopping the ingress of fluid into the pump chamber 1108, whilst still being free to move to let fluid egress from the chamber 1108, when the pressure in the pump chamber 1108 is sufficient to force the flange 122 to move and thus force the valve assembly 100 to open. When the pressure in the chamber 1108 is reduced the flange 122 returns to its position sealed over the entrance to the narrower portion 132 (e.g. by virtue of gravity) and prevents the flow of fluid into and out of the chamber 1108.

As outlined above, the one way valve position finds particular use in the pumping process that creates the reduced pressure in the chamber 1108 of the pump 1100.

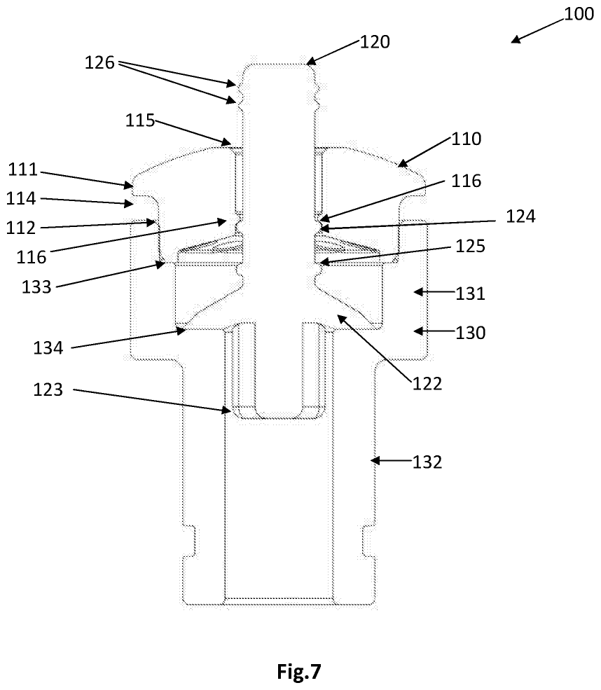

FIG. 7 shows the valve assembly 100 in a locked closed position. In this position, the first ridge 124 is disposed below projection 116, which holds the stem 120 in a position where the flange 122 seals over the entrance to the narrower portion 132. It will be appreciated that “below” in this context refers to the ridge 124 being closer to the pump chamber 1108 and narrower portion 132 than the projection 116.

The shape and flexible nature of the flange 122 means that the flange 122 deforms slightly when placed in this position, as the stem 120 is compressed between the projection 116 and lip 134. Accordingly, the tension in the flange 122 counteracts the vacuum pressure inside the pump chamber and creates a well formed positive seal to prevent the flow of fluid from passing in both directions. In addition, the properties of the material of the stem 120 and flange 122 are such that the stem acts as sprung tensioner, biased to counteract the downward force applied by the user to place it in the locked position, thus giving the valve assembly 100 positive locking feel and action.

The locked closed position may be particularly useful when the user wishes to fill the chamber 1108 with water, for example in the shower. The valve assembly 100 is locked closed, and the pump 1102 is inverted such that the base portion 1104 is above the head section 1106, so that the chamber 1108 acts as a cup to conveniently receive the water.

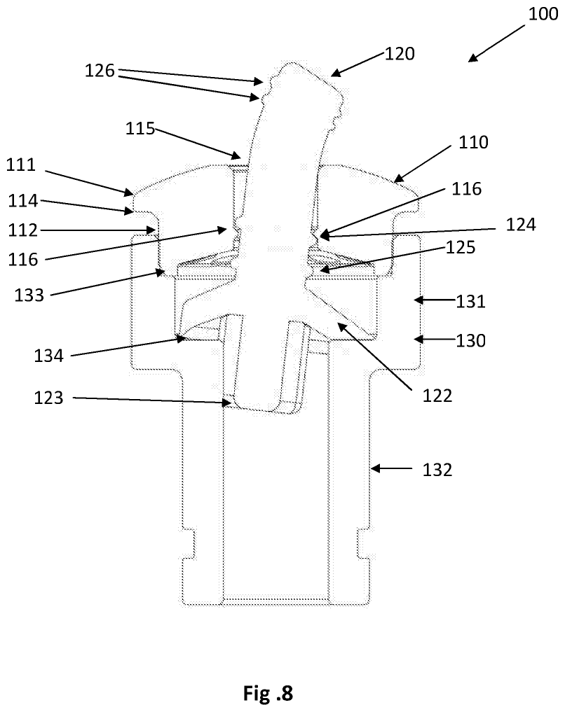

FIG. 8 shows a further function of the valve assembly 100. When the stem 120 is arranged in the one-way valve position, a lateral force F can be applied to the stem 120. By lateral force, it is meant a force that is substantially radial with respect to the longitudinal axis of the stem. The application of this force causes the flange 122 to become slightly unseated from its position covering the entrance to the narrower portion of the body 132. Accordingly, a small amount of fluid is permitted to egress the chamber 1108. This allows the valve assembly 100 to act as a slow release valve, and provides for an easy-to-operate means of adjusting the pressure in the chamber 1108. This slow release or bleed functionality is particularly effective in situations where the pump chamber 1108 is at a medium level of negative pressure/vacuum, because the pressure differential is not so high that the sudden release holds the valve assembly 100 open.

In use, the valve 100 is installed in the head section 1106. The body 130 of the valve 100 is securely retained in an accommodating recess in the head section 1106.

In one example, the end portion of the narrower portion 132 may extend through a hole (not shown) into the pump chamber 1108 and be sealed on the interior of the pump chamber 1108, for example by an O-ring (not shown) extending around the narrower portion 132. In such an example, the valve assembly 100 can be urged away from the pump chamber 1108, in order to effect the seal between chamber 1108 and valve body 130. For example, a resilient member (such as a spring, not shown) may be disposed to contact the interior of the accommodating recess and the bottom of wider portion 131. The skilled reader will appreciate that other means of holding the valve assembly 100 in place may be provided.

In order to use the valve assembly 100, the user moves the valve assembly 100 between the locked closed position and the one way valve position by applying a sufficient pulling force to move the first ridge 124 over the projection 116. A sufficient pushing force may be applied to move the valve assembly 100 from the one way valve position to the locked closed position. Similarly, the valve assembly 100 is moved between the one way valve position and the locked open position by applying a sufficient pulling force to move the second ridge 125 over the projection 116. A sufficient pushing force may be applied to move the valve assembly 100 from the locked open position to the one way valve position.

Accordingly, axial force alone can be used to select the position/function of the valve assembly 100.

Various modifications of the examples discussed herein within the scope of the invention will be apparent to the skilled reader.

In particular, in a further example of the valve assembly 100, the second ridge 126 formed on the stem 120 is omitted. This allows the valve to be placed in the locked closed position and the one way valve position, but not the locked open position. In this example, the motion of stem 120 in the one way valve position is limited by the first ridge 125 and the top of the flange 122, or another stopping element.

In a further example, the valve body 130 may be integrally formed with in the head section 1106 of the pump 1100.

The above-described valves have a structure that avoids 2-part over moulding of the stem, thereby significantly decreasing production and tooling costs. In addition, the above-described valves are advantageously easy to operate. In particular, the reliance on axial motion of the valve stem means that the user need not assess the orientation of the stem in order to manipulate it, as is the case in prior art valves which require lateral motion of the stem in a particular direction to lock/unlock the valve. Similarly, lateral force in any direction is able to activate the slow release function, thereby advantageously making the functionality easily accessible regardless of the orientation of the pump. Furthermore, the valves are advantageously able to function in various modes, improving user convenience.

Other advantageous aspects of the invention are outlined in the following numbered clauses:

1. A device for increasing the operating performance of hydropump penis pumps which makes the hydropump more operator friendly in terms of ease of use.
2. A device for increasing the operating performance of hydropump penis pumps which reduces production costs and tooling costs.
3. A device for increasing the operating performance of hydropump penis pumps and improving the serviceability and longer life of the device by minimising moving parts and design complexities.
4. A device for increasing the performance of Hydropump penis pumps and making the manufacture, assembly and inspection of the pumps much easier and more cost effective.
5. A device for increasing the operating performance of hydropump penis pumps and reducing materials and materials costs.
6. A device for increasing the operating performance of hydropump penis pumps by allowing superior operator control of vacuum pressure, and vacuum pressure release, by controlling the flow or air/fluid through the valve.
7. A device for increasing the operating performance of hydropump penis pumps which is operator friendly inasmuch as no technical training or technical knowledge is required.
8. A device for increasing the operating performance of hydropump penis pumps through three easy manual movements by the operator.
9. A device for increasing the performance of hydropump penis pumps using injection moulding techniques and standard materials to allow for mass production and affordability
10. A device which is self applied by the user and requires no technical knowledge or skills.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A valve assembly (100) for a penis pump (1102), comprising:
a valve cap (110) comprising a hole (115); and
a valve stem (120) extending through the hole (115) in the valve cap (110),
the valve assembly (100) being adapted such that axial motion of the valve stem (120) causes the valve stem (120) to mechanically interact with the valve cap (110), so that the valve stem (120) moves between:
a first position, in which the valve stem (120) is retained so as to seal a pump chamber (1108) of the penis pump (1102), and
a second position, in which the valve stem (120) operates as a one way valve.

2. The valve assembly of claim 1, wherein the valve assembly is configured to permit egress of fluid from the pump chamber in the second position.

3. The valve assembly of claim 2, wherein the valve stem comprises at least one stem projection formed thereon, wherein the at least one stem projection is configured to deform to allow the valve stem to move between the positions.

4. The valve assembly of claim 3, wherein the at least one stem projection is an annular ridge.

5. The valve assembly of claim 3, wherein the hole comprises a hole projection to mechanically interact with the at least one stem projection of the valve stem.

6. The valve assembly of claim 5, wherein the hole projection is annular.

7. The valve assembly of claim 5, wherein the at least one stem projection includes a first projection, and wherein:
in the first position, the first projection is disposed at a side of the hole projection that is proximal to the pump chamber, and
in the second position, the first projection may be disposed at side of the hole projection that is distal from the pump chamber.

8. The valve assembly of claim 1, wherein the valve assembly is configured such that axial motion of the valve stem causes the valve stem to move to a third position, in which the valve assembly is locked open.

9. The valve assembly of claim 6, wherein:
the valve assembly is configured such that axial motion of the valve stem causes the valve stem to move to a third position, in which the valve assembly is locked open;
the at least one stem projection include a second projection, the second projection being disposed closer to the pump chamber in use than the first projection, and
in the third position, the second projection is disposed at the side of the hole projection that is distal from the pump chamber.

10. The valve assembly of claim 1, wherein the valve assembly is configured so that, in the second position, receipt of a radial force applied to the stem causes the seal of pump chamber to be partially opened.

11. The valve assembly of claim 1, wherein the valve stem comprises a flange for sealing the pump chamber, extending radially from the valve stem.

12. The valve assembly of claim 1, wherein the valve stem is flexible.

13. The valve assembly of claim 1, wherein the valve cap is rigid.

14. The valve assembly of claim 1, comprising a valve body configured to retain the valve cap and the valve stem, wherein the valve body comprises:
a first portion arranged to receive the valve cap, and a second portion sealable by the valve stem.

15. A penis pump comprising the valve assembly claim 1.

16. A method of causing or enhancing an erection of a human penis comprising:
inserting a generally flaccid penis into a chamber of a penis pump and using pumping means to pump water from the chamber through the valve assembly of claim 1.
